(19) United States
(12) Patent Application Publication
(10) Pub. No.: US 2026/0301274 A1
(43) Pub. Date: Oct. 1, 2026

(54) IMAGE PROCESSING DEVICE AND IMAGE PROCESSING METHOD

(71) Applicant: HAMAMATSU PHOTONICS K.K., Hamamatsu-shi, Shizuoka (JP)

(72) Inventors: Kibo OTE, Shizuoka (JP); Fumio HASHIMOTO, Gainesville, FL (US); Yuya ONISHI, Shizuoka (JP)

(73) Assignee: HAMAMATSU PHOTONICS K.K., Hamamatsu-shi, Shizuoka (JP)

(21) Appl. No.: 19/477,608

(22) PCT Filed: Apr. 1, 2024
(86) PCT No.: PCT/JP2024/013502

§ 371 Date: Oct. 22, 2025

(30) Foreign Application Priority Data
Apr. 25, 2023 (JP) 2023-071328

(51) Int. Cl. G06T 12/20

(52) U.S. Cl. CPC G06T 12/20; G06T 2210/41; G06T 2211/441

(57) ABSTRACT

An image processing apparatus creates a measurement image in a measurement space different from a real space based on measurement data of a subject acquired by a measurement by using a tomography apparatus, inputs the measurement image to a CNN, and creates a tomographic image in the real space by the CNN. The CNN is based on a U-net structure including an encoder unit, a decoder unit, and a bottleneck unit, and further includes a projection connection unit. The projection connection unit performs projection processing from a sinogram space (the measurement space) to the real space on any one feature map in the encoder unit, and connects the feature map after performing the projection processing to the feature map of an equal size in the decoder unit.

### TECHNICAL FIELD

The present disclosure relates to an apparatus and a method for creating a tomographic image of a subject based on measurement data of the subject acquired by a measurement performed by using a tomography apparatus.

### BACKGROUND ART

Examples of a tomography apparatus capable of acquiring measurement data for creating a tomographic image of a subject include a positron emission tomography (PET) apparatus, a single photon emission computed tomography (SPECT) apparatus, an X-ray computed tomography (CT) apparatus, and a magnetic resonance imaging (MRI) apparatus.

The measurement data of the subject acquired by using each of the above tomography apparatuses do not directly represent the tomographic image of the subject in a real space without change. In order to create the tomographic image of the subject in the real space based on the measurement data of the subject acquired by using the tomography apparatus, first, a measurement image in a measurement space which is different from the real space is created based on the measurement data, and then, the tomographic image in the real space is created based on the measurement image in the measurement space. A health condition of the subject can be diagnosed based on the above tomographic image.

In the case of each of the PET apparatus, the SPECT apparatus, and the X-ray CT apparatus, the measurement space is set to a sinogram space, and the measurement image is set to a sinogram.

In the case of the three-dimensional PET apparatus, the sinogram is expressed as a histogram representing a frequency (a generation frequency of coincidence events) at which coincidence detection information is acquired, in the sinogram space represented by four variables of r, θ, z, and δ. The variable r represents a position in a radial direction of a coincidence detection line (a line connecting two detectors which perform coincidence detection of a photon pair). The variable θ represents an azimuth angle of the coincidence detection line. The variable z represents a position in a center axis direction of a midpoint of the coincidence detection line. Further, the variable 6 represents a polar angle of the coincidence detection line.

In the case of the MRI apparatus, the measurement space is set to a k space represented with a spatial frequency as a variable, and the measurement image is configured by k space data.

When creating the tomographic image in the real space based on the measurement image in the measurement space, image reconstruction processing is performed. As an image reconstruction method from the sinogram to the tomographic image, several techniques are known. For example, a maximum likelihood expectation maximization (ML-EM), an ordered subset expectation maximization (OSEM), a row action maximum likelihood algorithm (RAMLA), and a dynamic RAMLA (DRAMA), and the like are known.

In the above image reconstruction methods, forward projection from the real space to the sinogram space and back projection from the sinogram space to the real space are alternately and repeatedly performed, and thus, the reconstruction of the tomographic image is iteratively performed.

Further, a technique has also been proposed which uses a neural network to create the tomographic image in the real space from the measurement image in the measurement space. The above technique is expected to be capable of creating the tomographic image with higher performance. A technique described in Non Patent Document 1 is intended to input the sinogram to a convolutional neural network (CNN), and to create the tomographic image by the CNN.

### CITATION LIST

### Non Patent Literature

Non Patent Document 1: I. Haggstrom et al., “DeepPET: A deep encoder-decoder network for directly solving the PET image reconstruction inverse problem”, Med. Image Anal. Vol. 54, pp. 253-262, 2019

### SUMMARY OF INVENTION

### Technical Problem

In the technique described in Non Patent Document 1, it is required that the CNN is trained in advance by using many pairs of the sinograms and the tomographic images of the subjects. The input image to the CNN is the sinogram in the sinogram space, and on the other hand, the output image from the CNN is the tomographic image in the real space. The input image (the sinogram) and the output image (the tomographic image) are images of different types in the different spaces, and are significantly different in appearance. Therefore, the creation of the tomographic image based on the sinogram by using the CNN is a difficult task. Accordingly, in the technique described in Non Patent Document 1, a large amount of data (pairs of the sinograms and the tomographic images) is required for the training of the CNN.

In the case in which the amount of data used for the training is small, the CNN fails to learn an algorithm for creating the tomographic image from the sinogram, and merely stores the pairs of the sinograms and the tomographic images used for the training. In addition, when an unknown sinogram is input, the CNN outputs the tomographic image corresponding to the sinogram out of the sinograms used for the training which is closest to the input sinogram.

As described above, in the technique described in Non Patent Document 1, when the amount of data used for the training of the CNN is small, a performance of the creation of the tomographic image by using the CNN is low. In order to improve the performance of the creation of the tomographic image by the CNN, a large amount of data is required for performing the training of the CNN.

The problems when creating the tomographic image by using the CNN described above are present not only when creating the tomographic image from the sinogram in the PET apparatus, but also when creating the tomographic image in the real space from the measurement image in the measurement space in each of the SPECT apparatus, the X-ray CT apparatus, and the MRI apparatus.

An object of the present invention is to provide an image processing apparatus and an image processing method capable of suppressing an increase of an amount of data required for training of a CNN and improving a performance of creation of a tomographic image by the CNN.

### Solution to Problem

An embodiment of the present invention is an image processing apparatus. The image processing apparatus is an image processing apparatus for creating a tomographic image of a subject in a real space based on measurement data of the subject acquired by a measurement by using a tomography apparatus, and includes (1) a measurement image creation unit for creating a measurement image in a measurement space different from the real space based on the measurement data; and (2) a CNN processing unit for inputting the measurement image to a convolutional neural network, and creating the tomographic image by the convolutional neural network, and the convolutional neural network includes (a) an encoder unit for inputting the measurement image, updating a feature map by a convolution operation performed by each of a plurality of convolutional layers, and gradually decreasing a spatial size of the feature map; (b) a decoder unit provided at a subsequent stage of the encoder unit, and for updating the feature map by a convolution operation performed by each of a plurality of convolutional layers, and gradually increasing a spatial size of the feature map; and (c) a projection connection unit for performing projection processing from the measurement space to the real space on any one feature map in the encoder unit, and connecting the feature map after performing the projection processing to the feature map of an equal size in the decoder unit.

An embodiment of the present invention is a tomography system. The tomography system includes a tomography apparatus for acquiring measurement data for creating a tomographic image of a subject; and the image processing apparatus of the above configuration for creating the tomographic image based on the measurement data.

An embodiment of the present invention is an image processing method. The image processing method is an image processing method for creating a tomographic image of a subject in a real space based on measurement data of the subject acquired by a measurement by using a tomography apparatus, and includes (1) a measurement image creation step of creating a measurement image in a measurement space different from the real space based on the measurement data; and (2) a CNN processing step of inputting the measurement image to a convolutional neural network, and creating the tomographic image by the convolutional neural network, and the convolutional neural network includes (a) an encoder unit for inputting the measurement image, updating a feature map by a convolution operation performed by each of a plurality of convolutional layers, and gradually decreasing a spatial size of the feature map; (b) a decoder unit provided at a subsequent stage of the encoder unit, and for updating the feature map by a convolution operation performed by each of a plurality of convolutional layers, and gradually increasing a spatial size of the feature map; and (c) a projection connection unit for performing projection processing from the measurement space to the real space on any one feature map in the encoder unit, and connecting the feature map after performing the projection processing to the feature map of an equal size in the decoder unit.

### Advantageous Effects of Invention

According to the embodiments of the present invention, it is possible to suppress an increase of an amount of data required for training of a CNN, and improve a performance of creation of a tomographic image by the CNN.

### BRIEF DESCRIPTION OF DRAWINGS

FIG. 1 is a diagram illustrating a configuration of a tomography system 1.

FIG. 2 is a diagram illustrating a configuration example of a CNN.

FIG. 3 is a diagram for describing projection processing between a sinogram space (a measurement space) and a real space.

FIG. 4 is a diagram illustrating another configuration example of the CNN.

FIG. 5 includes (a), (b) diagrams showing tomographic images in the case in which a sample 1 without noise is used in a comparative example.

FIG. 6 includes (a), (b) diagrams showing tomographic images in the case in which a sample 19 without noise is used in the comparative example.

FIG. 7 includes (a), (b) diagrams showing tomographic images in the case in which the sample 1 without noise is used in an example.

FIG. 8 includes (a), (b) diagrams showing tomographic images in the case in which the sample 19 without noise is used in the example.

FIG. 9 includes (a), (b) diagrams showing tomographic images in the case in which the sample 1 with noise is used in the comparative example.

FIG. 10 includes (a), (b) diagrams showing tomographic images in the case in which the sample 19 with noise is used in the comparative example.

FIG. 11 includes (a), (b) diagrams showing tomographic images in the case in which the sample 1 with noise is used in the example.

FIG. 12 includes (a), (b) diagrams showing tomographic images in the case in which the sample 19 with noise is used in the example.

### DESCRIPTION OF EMBODIMENTS

Hereinafter, embodiments of an image processing apparatus and an image processing method will be described in detail with reference to the accompanying drawings. In the description of the drawings, the same elements will be denoted by the same reference signs, and redundant description will be omitted. The present invention is not limited to these examples, and the Claims, their equivalents, and all the changes within the scope are intended as would fall within the scope of the present invention.

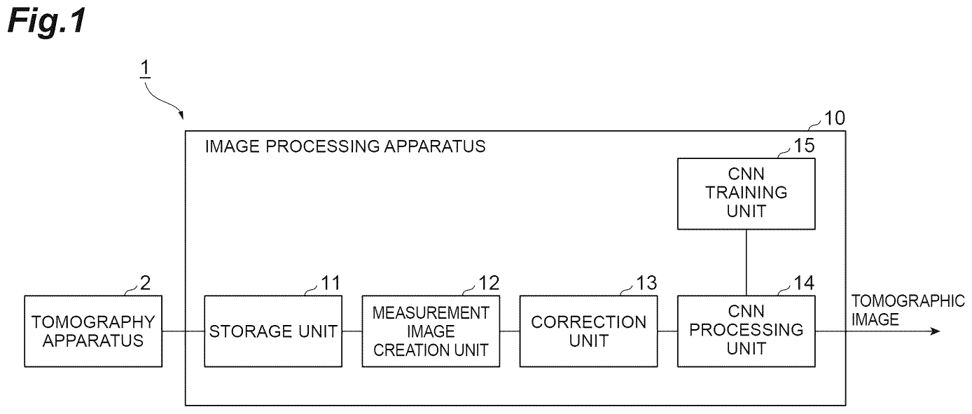

FIG. 1 is a diagram illustrating a configuration of a tomography system 1. The tomography system 1 is a system for creating a tomographic image of a subject, and includes a tomography apparatus 2 and an image processing apparatus 10. The tomography apparatus 2 is an apparatus for acquiring measurement data used for creating the tomographic image of the subject, and is, for example, a PET apparatus, a SPECT apparatus, an X-ray CT apparatus, or an MRI apparatus.

The image processing apparatus 10 includes a storage unit 11, a measurement image creation unit 12, a correction unit 13, a CNN processing unit 14, and a CNN training unit 15. An image processing method includes a storage step, a measurement image creation step, a correction step, a CNN processing step, and a CNN training step.

The image processing apparatus 10 creates the tomographic image of the subject in a real space based on the measurement data of the subject acquired by a measurement by using the tomography apparatus 2. The image processing apparatus 10 includes an operation processing unit including a CPU or a GPU for performing processing of various types, a storage unit including a hard disk drive, a RAM, and the like for storing a program and data for performing the processing, a display unit such as a liquid crystal display for displaying a processing condition, a processing result, and the like, and an input unit including a keyboard, a mouse, and the like for receiving input of the processing condition and the like. The image processing apparatus 10 may be configured by a computer.

In addition to the storage unit 11 for storing the program and the data, the image processing apparatus 10 includes, as the operation processing unit for performing the various types of processing, the measurement image creation unit 12, the correction unit 13, the CNN processing unit 14, and the CNN training unit 15. In the storage step, the storage unit 11 inputs the measurement data of the subject acquired by the measurement performed by using the tomography apparatus 2, and stores the measurement data.

In the measurement image creation step, the measurement image creation unit 12 creates a measurement image in a measurement space different from the real space based on the measurement data which is stored by the storage unit 11. In the correction step, the correction unit 13 performs various corrections (for example, sensitivity correction, absorptive correction, and scattering correction) on the measurement image which is created by the measurement image creation unit 12.

In the CNN processing step, the CNN processing unit 14 inputs the measurement image in the measurement space after being corrected by the correction unit 13 to a convolutional neural network (CNN), and creates the tomographic image in the real space by the CNN. In the CNN training step, the CNN training unit 15 trains the CNN by using the measurement image and the tomographic image of each of a plurality of subjects. The image processing apparatus 10 after the training of the CNN may not include the CNN training unit 15.

In the case in which the tomography apparatus 2 is the PET apparatus, the SPECT apparatus, or the X-ray CT apparatus, the measurement space is set to a sinogram space, and the measurement image is set to a sinogram. In the case in which the tomography apparatus 2 is the MRI apparatus, the measurement space is set to a k space, and the measurement image is configured by k space data.

In the following description, it is assumed that the tomography apparatus 2 is the PET apparatus. The PET apparatus is a radiation tomography apparatus capable of acquiring the tomographic image of the subject (a living body). The PET apparatus includes a detection unit having a large number of small radiation detectors which are arranged around a measurement space in which the subject is placed.

The PET apparatus detects a y-ray photon pair of an energy of 511 keV generated by the electron positron annihilation in the subject into which a positron-emitting isotope (an RI source) is injected by a coincidence method by using the detection unit, and collects the coincidence detection information. The above coincidence detection information includes position information of each of two radiation detectors which perform the coincidence detection of the photon pair, and a coincidence detection line (a line connecting the two radiation detectors which perform the coincidence detection of the photon pair) can be obtained from the position information. The storage unit 11 stores the coincidence detection information which is collected by using the PET apparatus.

The measurement image creation unit 12 creates the sinogram based on the collected coincidence detection information which is stored by the storage unit 11. The sinogram (the measurement image) is expressed as a histogram representing a frequency (a generation frequency of coincidence events) at which the coincidence detection information is acquired, in the sinogram space (the measurement space) in which a position, a direction, and the like of the coincidence detection line are set to be variables. The CNN processing unit 14 inputs the sinogram (the measurement image) in the sinogram space (the measurement space) to the CNN, and creates the tomographic image in the real space by using the CNN.

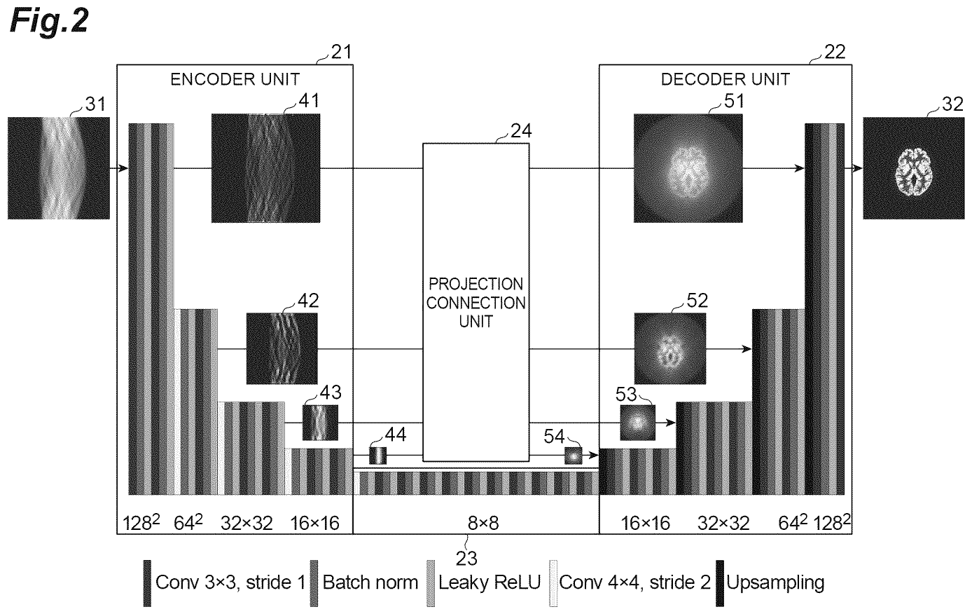

FIG. 2 is a diagram illustrating a configuration example of the CNN. The CNN illustrated in this diagram is based on a U-net structure including an encoder unit 21, a decoder unit 22, and a bottleneck unit 23, and further includes a projection connection unit 24.

In this diagram, feature maps after respective processing steps of a convolutional layer (Conv 3×3, stride 1), a normalization layer (Batch Normalization), a nonlinear activation function (Leaky ReLU), a downsampling (Conv 4×4, stride 2), and an upsampling (Upsampling) are illustrated by grayscale different from each other. Further, in this diagram, with the numbers of pixels of each of a sinogram 31 input to the CNN and a tomographic image 32 output from the CNN being set to 128×128, sizes of the respective feature maps are also illustrated.

The convolutional layer relatively moves a filter with respect to the feature map (or the input image) which is created beforehand, performs a convolution operation of the feature map and the filter at each position, and creates a new feature map of the same spatial size. The normalization layer normalizes the feature for each mini batch of the feature map. The nonlinear activation function inputs a weighted sum z of each value of the feature map to an activation function σ(z)=max{0, z}, and outputs a function value σ(z).

The downsampling performs the convolution operation of the feature map and the filter, decreases the spatial size of the feature map by ½×½, and increases the number of channels of the feature map by two times. The upsampling increases the spatial size of the feature map by 2×2 times by using linear interpolation, performs the convolution operation of the feature map and the filter, and decreases the number of channels of the feature map by ½.

The encoder unit 21 includes the convolutional layer, the normalization layer, the nonlinear activation function, and the downsampling. The encoder unit 21 inputs the sinogram (the measurement image) in the sinogram space (the measurement space), and updates the feature map by the convolutional layer, the normalization layer, and the nonlinear activation function. Further, after performing the processing steps by the convolutional layer, the normalization layer, and the nonlinear activation function several times, the encoder unit 21, by the downsampling, decreases the spatial size of the feature map by ½×½, and increases the number of channels of the feature map by two times. In this diagram, the spatial size of the feature map is initially set to 128×128, and then decreases to 64×64, 32×32, and 16×16.

The decoder unit 22 includes the convolutional layer, the normalization layer, the nonlinear activation function, and the upsampling. The decoder unit 22 is provided at the subsequent stage of the encoder unit 21, and by the upsampling, increases the spatial size of the feature map by two times, and decreases the number of channels of the feature map by ½×½. In this diagram, the spatial size of the feature map increases to 16×16, 32×32, 64×64, and 128×128. Further, after performing the processing step by the upsampling, the decoder unit 22 updates the feature map by the convolutional layer, the normalization layer, and the nonlinear activation function.

The bottleneck unit 23 is provided between the encoder unit 21 and the decoder unit 22. The bottleneck unit 23 includes the convolutional layer, the normalization layer, and the nonlinear activation function, and updates the feature map by these. In the bottleneck unit 23, while maintaining the spatial size of the feature map, the number of channels is increased by two times.

The encoder unit 21 inputs the sinogram (the measurement image) in the sinogram space (the measurement space), and extracts the feature of the input sinogram as the feature map. On the other hand, the decoder unit 22 gradually reconstructs the tomographic image in the real space based on the feature map indicating the extracted feature of the sinogram.

The projection connection unit 24 performs projection processing from the sinogram space (the measurement space) to the real space on any one feature map in the encoder unit 21, and connects the feature map after performing the projection processing to the feature map of the equal size in the decoder unit 22. The projection connection unit 24 may perform the projection processing on any arbitrary feature map in the encoder unit 21, and in addition, it is preferable to perform the projection processing on the feature map of the maximum size in the encoder unit 21, and it is more preferable to perform the projection processing on the feature maps of all sizes in the encoder unit 21.

In any of the cases, the spatial size of the feature map is not changed by the projection processing, and the feature map after performing the projection processing is connected to the feature map of the same spatial size in the decoder unit 22. The connection of the feature map after performing the projection processing to the feature map in the decoder unit may be performed by addition for each channel, or may be performed by stacking of channels. For the feature map of the spatial size on which the projection connection is not performed, the feature map without change may be connected (a skip connection) to the feature map of the same spatial size in the decoder unit 22.

In the configuration of FIG. 2, the projection connection unit 24 performs the projection processing on the feature maps of all sizes in the encoder unit 21, and connects the feature maps after performing the projection processing respectively to the feature maps of the equal sizes in the decoder unit 22.

That is, the projection processing is performed on the feature map 41 of the size of 128×128 in the encoder unit 21, and the feature map 51 after performing the projection processing is connected to the feature map of the size of 128×128 in the decoder unit 22. The projection processing is performed on the feature map 42 of the size of 64×64 in the encoder unit 21, and the feature map 52 after performing the projection processing is connected to the feature map of the size of 64×64 in the decoder unit 22.

The projection processing is performed on the feature map 43 of the size of 32×32 in the encoder unit 21, and the feature map 53 after performing the projection processing is connected to the feature map of the size of 32×32 in the decoder unit 22. The projection processing is performed on the feature map 44 of the size of 16×16 in the encoder unit 21, and the feature map 54 after performing the projection processing is connected to the feature map of the size of 16×16 in the decoder unit 22.

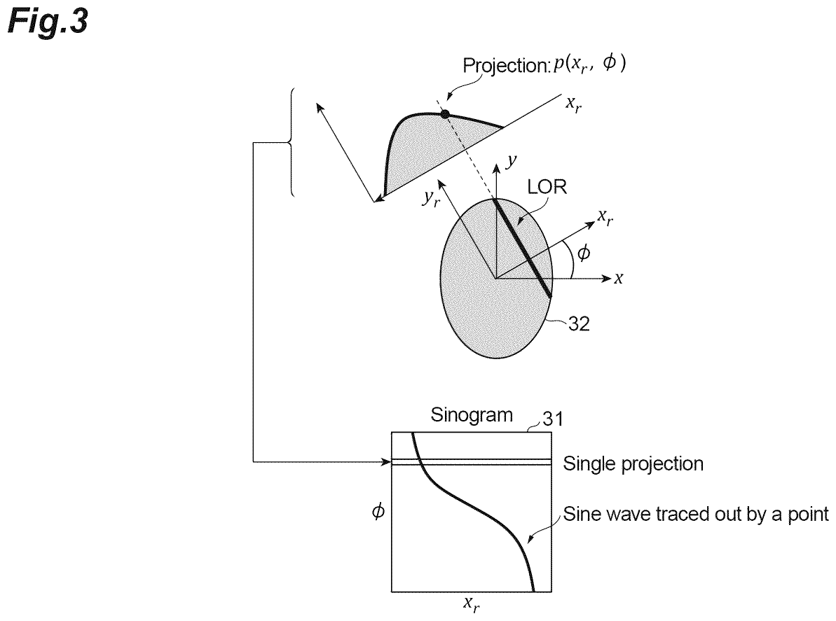

FIG. 3 is a diagram for describing the projection processing between the sinogram space (the measurement space) and the real space. This diagram illustrates a relationship between the sinogram 31 and the tomographic image 32 in the two-dimensional PET apparatus. In the iterative image reconstruction method, projection from the real space to the sinogram space is referred to as forward projection, and projection from the sinogram space to the real space is referred to as back projection. The iterative image reconstruction method alternately and repeatedly performs the forward projection and the back projection to iteratively reconstruct the tomographic image.

The forward projection is processing for transforming the tomographic image 32 in the real space into the sinogram 31, and is represented by the following Formula (1). p is the sinogram, and f is the tomographic image. A is a system matrix representing an operation of the forward projection. As illustrated in the diagram, the forward projection is the processing for obtaining, for the coincidence detection line (line of response, LOR) of each position xᵣ and each direction φ, a sum p(xᵣ, φ) of pixel values of pixels through which the coincidence detection line passes in the tomographic image 32.

$$\begin{matrix}[ Formula 1 ] & \\ p = Af & ( 1 )\end{matrix}$$

The forward projection is analytically represented by the following Formula (2). In this case, there is a relationship represented by the following Formula (3) between a coordinate (x, y) and a coordinate (xᵣ, yᵣ). φ is an azimuth angle representing a direction of the projection. xᵣ and yᵣ are an x axis and a y axis in the case in which the tomographic image is rotated by −φ. The forward projection obtains, after rotating the image by −φ degrees, the one-dimensional projection p(xᵣ, φ) in the direction of φ degrees by integrating the pixel values along the yᵣ axis, and by repeatedly performing the above operation for 0≤φ<π, the sinogram 31 is obtained.

$$\begin{matrix}[ Formula 2 ] & \\ p ( {x}_{r} , ϕ ) = {\int }_{- ∞}^{∞} {dy}_{r} f ( x , y ) & ( 2 )\end{matrix}$$

$$\begin{matrix}[ Formula 3 ] & \\ [ \begin{matrix}x \\ y\end{matrix} ] = [ \begin{matrix}cos ϕ & - sin ϕ \\ sin ϕ & cos ϕ\end{matrix} ] [ \begin{matrix}{x}_{r} \\ {y}_{r}\end{matrix} ] & ( 3 )\end{matrix}$$

The back projection is processing for projecting the sinogram 31 onto the real space by using a transposed matrix A^{T} of the matrix A, and is represented by the following Formula (4). b is aback projection image of the sinogram p. The back projection image b is not equal to the tomographic image f.

$$\begin{matrix}[ Formula 4 ] & \\ b = {A}^{T} p & ( 4 )\end{matrix}$$

The projection processing performed by the projection connection unit 24 (the projection processing from the sinogram space to the real space on any feature map in the encoder unit 21) corresponds to the back projection described above. By providing the above projection connection unit 24, it is expected that the processing on the sinogram is learned in the encoder unit 21, and it is expected that the processing on the tomographic image is learned in the decoder unit 22. In addition, it is expected that learning of the algorithm for creating the tomographic image from the sinogram is promoted without being confined to storing of the pairs of the sinograms and the tomographic images used for the training.

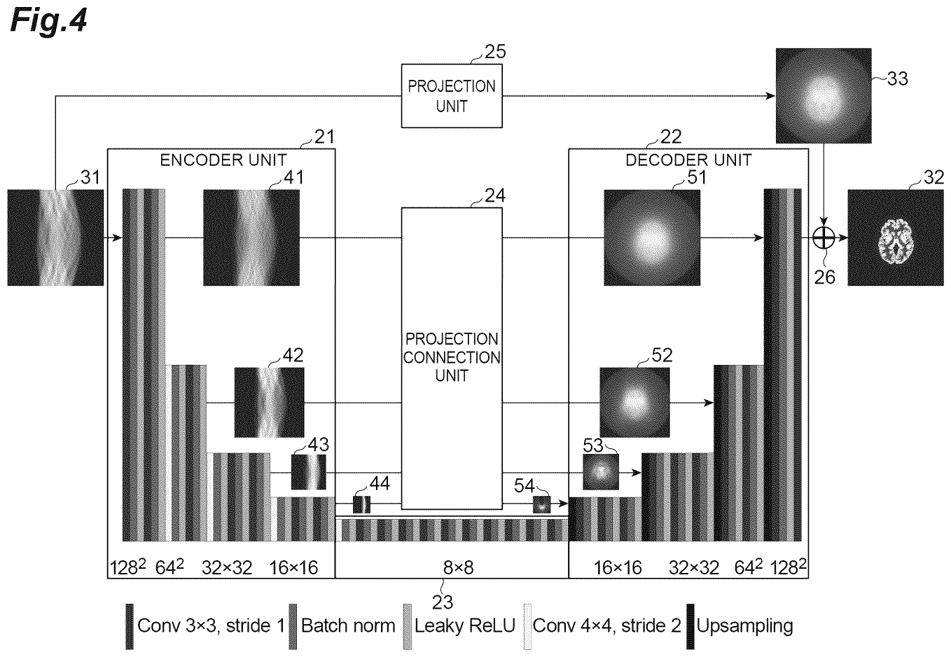

FIG. 4 is a diagram illustrating another configuration example of the CNN. As compared with the CNN illustrated in FIG. 2, the CNN illustrated in this diagram is different in that the CNN further includes a projection unit 25 and an addition unit 26. The projection unit 25 performs the projection processing (the back projection) from the sinogram space to the real space on the sinogram (the measurement image) 31 input to the encoder unit 21, and creates a projection image 33 which is an image after performing the projection. The addition unit 26 adds the projection image 33 to the output image from the decoder unit 22 to create the tomographic image 32.

In the case of using the above configuration, when the CNN training unit 15 trains the CNN by using the sinogram 31 and the tomographic image 32 of each of the plurality of subjects, the CNN is made to learn (residual learning) a difference between the projection image 33 obtained by the back projection of the sinogram 31 and the tomographic image 32. The difference for each pixel between the projection image 33 and the tomographic image 32 can be a positive value, and further, can be a negative value, and has a distribution which can be approximated by a normal distribution centered at a value of 0. Accordingly, the training of the CNN by using a least squares method becomes easier.

Next, simulation results obtained by creating simulation data by numerical simulation by using a digital brain phantom image will be described. A case of using the CNN illustrated in FIG. 2 was set to an example. A technique of creating the tomographic image from the histogram by using the CNN which is described in Non Patent Document 1 was set to a comparative example. In the configuration of the comparative example, a unit corresponding to the projection connection unit 24 is not provided in the CNN.

MRI brain segmentation images of 20 samples were downloaded from BrainWeb (https://brainweb.bic.mni.mcgill.ca/brainweb/), and brain phantom images of the PET simulating ¹⁸F-FDG (¹⁸F-fluorodeoxyglucose) as a drug were created with a contrast ratio of gray matter:white matter:cerebrospinal fluid (CSF) set to 1:0.25:0.05. A matrix size of each phantom image was set to 128 (X)×128 (Y)×70 (Z). A sinogram was created by performing the forward projection on each phantom image. A matrix size of each sinogram was set to 128 (Xᵣ)×128 (φ)×70 (Z).

For each sample, two types of one without noise and one with noise were prepared. A sinogram with noise was created by adding Poisson noise corresponding to a case in which a total count for one sample is set to 10 M. Out of the pairs of the sinograms and the tomographic images of the 20 samples (70 slices for each sample), 18 samples were used as training data (a total of 1,260 slices), and 2 samples were used as test data (a total of 140 slices).

For each of the case of using the samples without noise and the case of using the samples with noise, after training the CNN by using the training data (the samples 1 to 18), the sinograms of the test data (the samples 19 and 20) were input to the CNN to create the tomographic images by using the CNN, and the created tomographic images were compared with the original tomographic images. The tomographic image which is created by the CNN was evaluated by using a value of a peak signal to noise ratio. The peak signal to noise ratio (PSNR) represents quality of the image by decibels (dB), and the higher value means the better image quality.

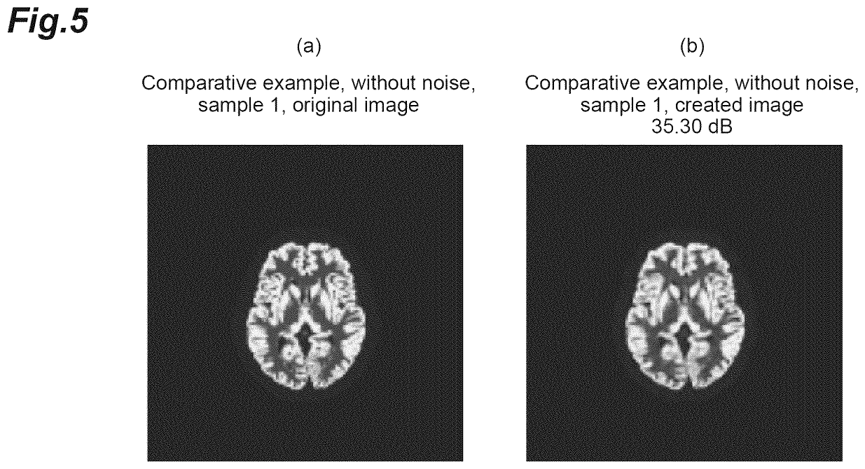

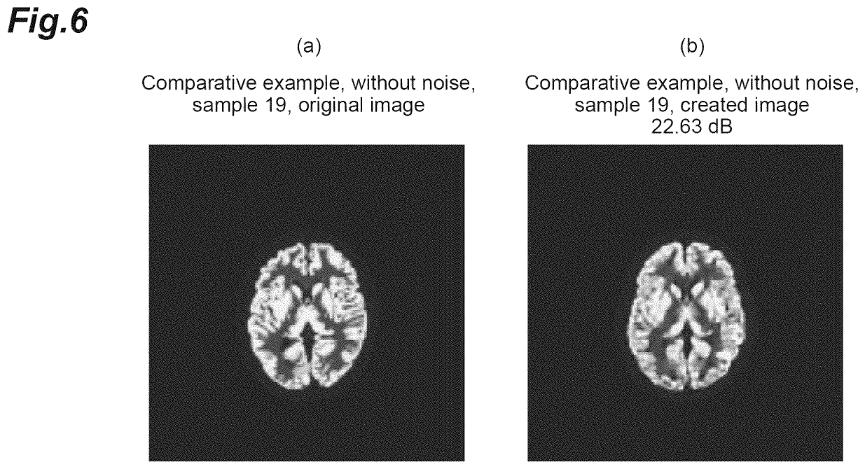

FIG. 5 and FIG. 6 include diagrams showing the tomographic images in the case in which the samples without noise are used in the comparative example. (a) in FIG. 5 is a diagram showing the original tomographic image of the sample 1. (b) in FIG. 5 is a diagram showing the tomographic image created by the CNN based on the sinogram of the sample 1. (a) in FIG. 6 is a diagram showing the original tomographic image of the sample 19. (b) FIG. 6 is a diagram showing the tomographic image created by the CNN based on the sinogram of the sample 19.

The PSNR of the tomographic image ((b) in FIG. 5) created by the CNN based on the sinogram of the training data (the sample 1) is 35.30 dB. The PSNR of the tomographic image ((b) in FIG. 6) created by the CNN based on the sinogram of the test data (the sample 19) is 22.63 dB.

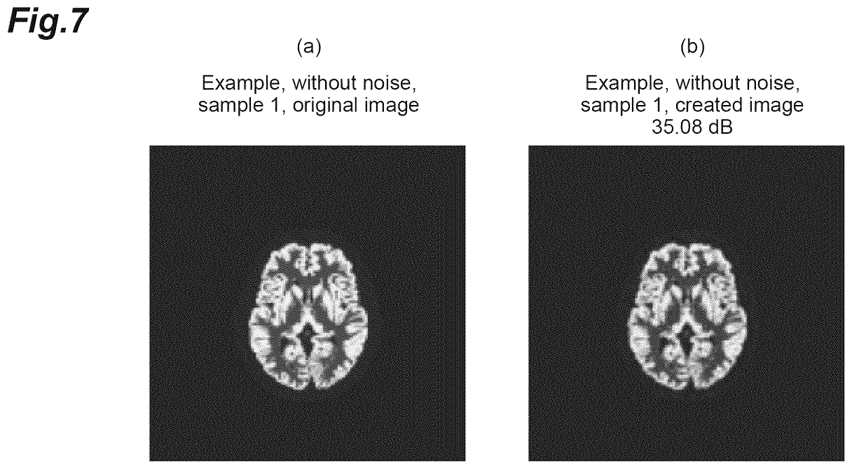

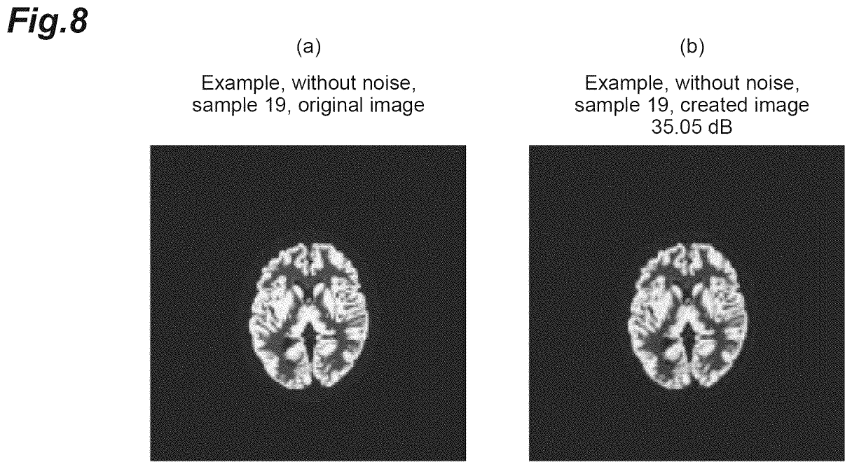

FIG. 7 and FIG. 8 include diagrams showing the tomographic images in the case in which the samples without noise are used in the example. (a) in FIG. 7 is a diagram showing the original tomographic image of the sample 1. (b) in FIG. 7 is a diagram showing the tomographic image created by the CNN based on the sinogram of the sample 1. (a) in FIG. 8 is a diagram showing the original tomographic image of the sample 19. (b) in FIG. 8 is a diagram showing the tomographic image created by the CNN based on the sinogram of the sample 19.

The PSNR of the tomographic image ((b) in FIG. 7) created by the CNN based on the sinogram of the training data (the sample 1) is 35.08 dB. The PSNR of the tomographic image ((b)) in FIG. 8) created by the CNN based on the sinogram of the test data (the sample 19) is 35.05 dB.

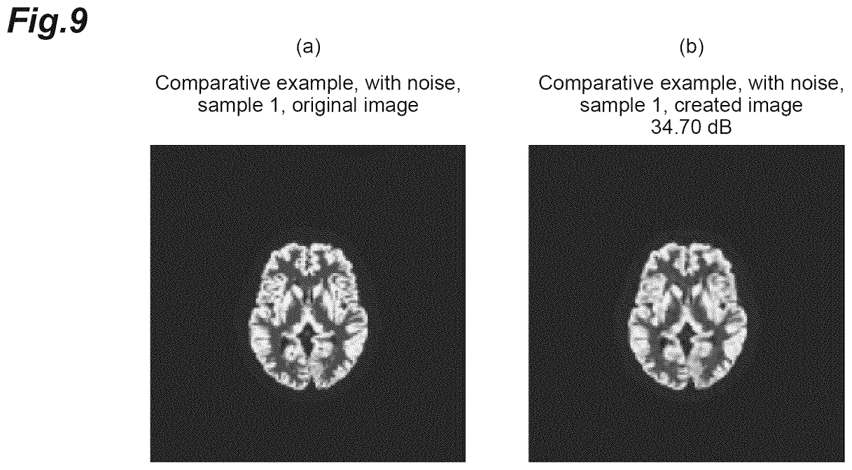

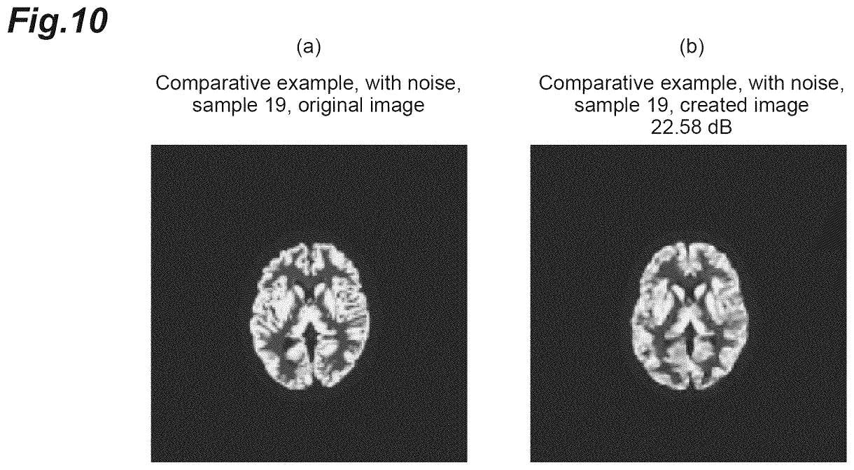

FIG. 9 and FIG. 10 include diagrams showing the tomographic images in the case in which the samples with noise are used in the comparative example. (a) in FIG. 9 is a diagram showing the original tomographic image of the sample 1. (b) in FIG. 9 is a diagram showing the tomographic image created by the CNN based on the sinogram of the sample 1. (a) in FIG. 10 is a diagram showing the original tomographic image of the sample 19. (b) in FIG. 10 is a diagram showing the tomographic image created by the CNN based on the sinogram of the sample 19.

The PSNR of the tomographic image ((b) in FIG. 9) created by the CNN based on the sinogram of the training data (the sample 1) is 34.70 dB. The PSNR of the tomographic image ((b) in FIG. 10) created by the CNN based on the sinogram of the test data (the sample 19) is 22.58 dB.

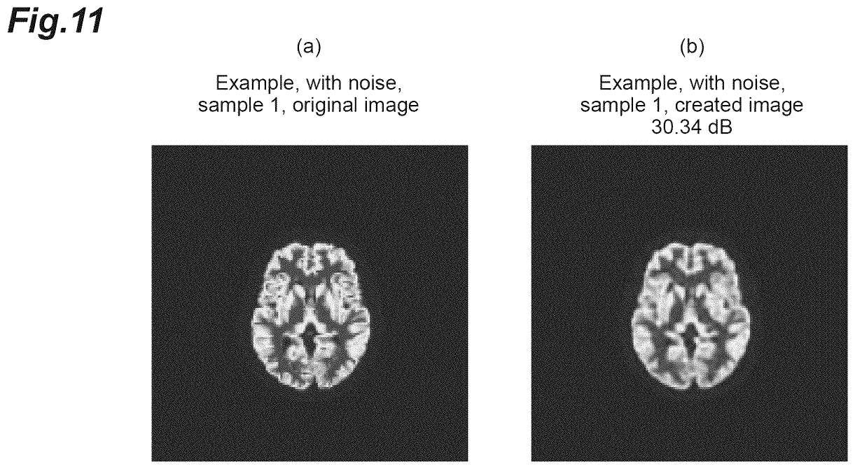

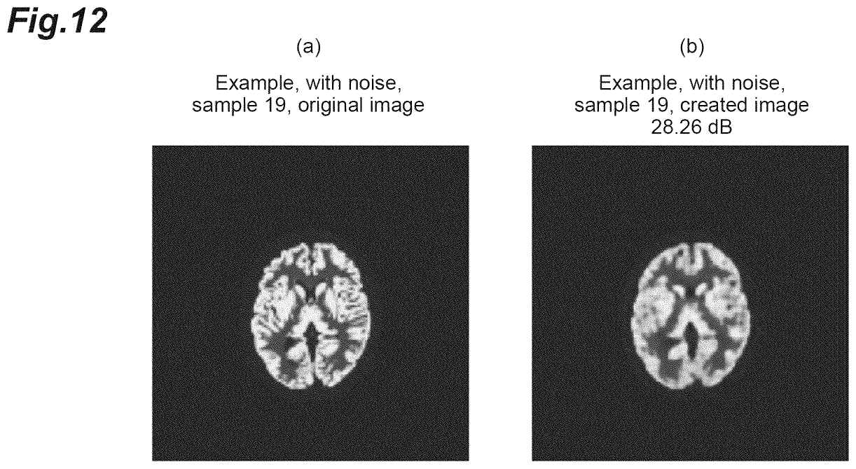

FIG. 11 and FIG. 12 include diagrams showing the tomographic images in the case in which the samples with noise are used in the example. (a) in FIG. 11 is a diagram showing the original tomographic image of the sample 1. (b) in FIG. 11 is a diagram showing the tomographic image created by the CNN based on the sinogram of the sample 1. (a) in FIG. 12 is a diagram showing the original tomographic image of the sample 19. (b) in FIG. 12 is a diagram showing the tomographic image created by the CNN based on the sinogram of the sample 19.

The PSNR of the tomographic image ((b) in FIG. 11) created by the CNN based on the sinogram of the training data (the sample 1) is 30.34 dB. The PSNR of the tomographic image ((b) in FIG. 12) created by the CNN based on the sinogram of the test data (the sample 19) is 28.26 dB.

By comparing the PSNR values of the respective tomographic images shown in FIG. 5 to FIG. 12, the following can be understood. In the case in which the tomographic image is created by the CNN based on the sinogram of the training data, regardless of the presence or absence of noise, the PSNR of the tomographic image created in the comparative example is as high as the PSNR of the tomographic image created in the example. On the other hand, in the case in which the tomographic image is created by the CNN based on the sinogram of the test data, regardless of the presence or absence of noise, the PSNR of the tomographic image created in the comparative example is low. This is considered to be due to the following reason.

It is considered that the CNN used in the comparative example merely stores the pairs of the sinograms and the tomographic images of the training data, and when the unknown sinogram of the test data is input, the CNN outputs the tomographic image corresponding to the sinogram out of the sinograms used for the training which is closest to the input sinogram. Therefore, in the comparative example, it is considered that generalization to unknown test data cannot be achieved, and the tomographic image of a different person is output.

On the other hand, in the example, not only when the tomographic image is created by the CNN based on the sinogram of the training data, but also when the tomographic image is created by the CNN based on the sinogram of the test data, regardless of the presence or absence of noise, the PSNR of the tomographic image created by the CNN is high. In the example, generalization to unknown test data is achieved very well. This is considered to be due to the following reason.

In the example, the CNN is provided with the projection connection unit 24, and thus, it is considered that the CNN is able to learn the algorithm for creating the tomographic image from the sinogram, without merely storing the pairs of the sinograms and the tomographic images used for the training.

As described above, according to the present embodiment, it is possible to suppress an increase in an amount of data required for the training of the CNN, and improve the performance of the creation of the tomographic image by the CNN.

In the embodiments and the examples described above, the case in which the tomography apparatus is set to the PET apparatus is mainly described. In addition, the present invention can be similarly applied to other tomography apparatuses.

In the case in which the tomography apparatus is the SPECT apparatus or the X-ray CT apparatus, as in the case of the PET apparatus, the measurement space is set to the sinogram space, the measurement image is set to the sinogram, and the projection processing from the measurement space to the real space is set to the back projection. In the case in which the tomography apparatus is the MRI apparatus, the measurement space is set to the k space, the measurement image is set to the image configured by the k space data, and the projection processing from the measurement space to the real space is set to inverse Fourier transform.

The image processing apparatus and the image processing method are not limited to the embodiments and configuration examples described above, and various modifications are possible.

The image processing apparatus of a first aspect according to the above embodiment is an image processing apparatus for creating a tomographic image of a subject in a real space based on measurement data of the subject acquired by a measurement by using a tomography apparatus, and includes (1) a measurement image creation unit for creating a measurement image in a measurement space different from the real space based on the measurement data; and (2) a CNN processing unit for inputting the measurement image to a convolutional neural network, and creating the tomographic image by the convolutional neural network, and the convolutional neural network includes (a) an encoder unit for inputting the measurement image, updating a feature map by a convolution operation performed by each of a plurality of convolutional layers, and gradually decreasing a spatial size of the feature map; (b) a decoder unit provided at a subsequent stage of the encoder unit, and for updating the feature map by a convolution operation performed by each of a plurality of convolutional layers, and gradually increasing a spatial size of the feature map; and (c) a projection connection unit for performing projection processing from the measurement space to the real space on any one feature map in the encoder unit, and connecting the feature map after performing the projection processing to the feature map of an equal size in the decoder unit.

In the image processing apparatus of a second aspect, in the configuration of the first aspect, the apparatus may further include a CNN training unit for training the convolutional neural network by using the measurement image and the tomographic image of each of a plurality of subjects.

In the image processing apparatus of a third aspect, in the configuration of the first or second aspect, the CNN processing unit may perform the projection processing from the measurement space to the real space on the measurement image input to the encoder unit, and may add an image after performing the projection processing to an output image from the decoder unit.

In the image processing apparatus of a fourth aspect, in the configuration of any one of the first to third aspects, the projection connection unit may perform the projection processing from the measurement space to the real space on the feature map of a maximum size in the encoder unit, and may connect the feature map after performing the projection processing to the feature map of an equal size in the decoder unit.

In the image processing apparatus of a fifth aspect, in the configuration of any one of the first to third aspects, the projection connection unit may performs the projection processing from the measurement space to the real space on each of the feature maps of all sizes in the encoder unit, and may connect the feature map after performing the projection processing to the feature map of an equal size in the decoder unit.

The tomography system according to the above embodiment includes a tomography apparatus for acquiring measurement data for creating a tomographic image of a subject; and the image processing apparatus of the above configuration for creating the tomographic image based on the measurement data.

The image processing method of a first aspect according to the above embodiment is an image processing method for creating a tomographic image of a subject in a real space based on measurement data of the subject acquired by a measurement by using a tomography apparatus, and includes (1) a measurement image creation step of creating a measurement image in a measurement space different from the real space based on the measurement data; and (2) a CNN processing step of inputting the measurement image to a convolutional neural network, and creating the tomographic image by the convolutional neural network, and the convolutional neural network includes (a) an encoder unit for inputting the measurement image, updating a feature map by a convolution operation performed by each of a plurality of convolutional layers, and gradually decreasing a spatial size of the feature map; (b) a decoder unit provided at a subsequent stage of the encoder unit, and for updating the feature map by a convolution operation performed by each of a plurality of convolutional layers, and gradually increasing a spatial size of the feature map; and (c) a projection connection unit for performing projection processing from the measurement space to the real space on any one feature map in the encoder unit, and connecting the feature map after performing the projection processing to the feature map of an equal size in the decoder unit.

In the image processing method of a second aspect, in the configuration of the first aspect, the method may further include a CNN training step of training the convolutional neural network by using the measurement image and the tomographic image of each of a plurality of subjects.

In the image processing method of a third aspect, in the configuration of the first or second aspect, in the CNN processing step, the projection processing from the measurement space to the real space may be performed on the measurement image input to the encoder unit, and an image after performing the projection processing may be added to an output image from the decoder unit.

In the image processing method of a fourth aspect, in the configuration of any one of the first to third aspects, the projection connection unit may perform the projection processing from the measurement space to the real space on the feature map of a maximum size in the encoder unit, and may connect the feature map after performing the projection processing to the feature map of an equal size in the decoder unit.

In the image processing method of a fifth aspect, in the configuration of any one of the first to third aspects, the projection connection unit may perform the projection processing from the measurement space to the real space on each of the feature maps of all sizes in the encoder unit, and may connect the feature map after performing the projection processing to the feature map of an equal size in the decoder unit.

### INDUSTRIAL APPLICABILITY

The present invention can be used as an image processing apparatus and an image processing method capable of suppressing an increase of an amount of data required for training of a CNN and improving a performance of creation of a tomographic image by the CNN.

### REFERENCE SIGNS LIST

1—tomography system, 2—tomography apparatus, 10—image processing apparatus, 11—storage unit, 12—measurement image creation unit, 13—correction unit, 14—CNN processing unit, 15—CNN training unit, 21—encoder unit, 22—decoder unit, 23—bottleneck unit, 24—projection connection unit, 25—projection unit, 26—addition unit, 31—sinogram, 32—tomographic image, 33—projection image, 41—44—feature map, 51—54—feature map.

1: An image processing apparatus for creating a tomographic image of a subject in a real space based on measurement data of the subject acquired by a measurement by using a tomography apparatus, the image processing apparatus comprising:
a measurement image creation unit configured to create a measurement image in a measurement space different from the real space based on the measurement data; and
a CNN processing unit configured to input the measurement image to a convolutional neural network, and create the tomographic image by the convolutional neural network, wherein
the convolutional neural network includes:
an encoder unit configured to input the measurement image, update a feature map by a convolution operation performed by each of a plurality of convolutional layers, and gradually decrease a spatial size of the feature map;
a decoder unit provided at a subsequent stage of the encoder unit, and configured to update the feature map by a convolution operation performed by each of a plurality of convolutional layers, and gradually increase a spatial size of the feature map; and
a projection connection unit configured to perform projection processing from the measurement space to the real space on any one feature map in the encoder unit, and connect the feature map after performing the projection processing to the feature map of an equal size in the decoder unit.

2: The image processing apparatus according to claim 1, further comprising:
a CNN training unit configured to train the convolutional neural network by using the measurement image and the tomographic image of each of a plurality of subjects.

3: The image processing apparatus according to claim 1, wherein the CNN processing unit is configured to perform the projection processing from the measurement space to the real space on the measurement image input to the encoder unit, and add an image after performing the projection processing to an output image from the decoder unit.

4: The image processing apparatus according to claim 1, wherein the projection connection unit is configured to perform the projection processing from the measurement space to the real space on the feature map of a maximum size in the encoder unit, and connect the feature map after performing the projection processing to the feature map of an equal size in the decoder unit.

5: The image processing apparatus according to claim 1, wherein the projection connection unit is configured to perform the projection processing from the measurement space to the real space on each of the feature maps of all sizes in the encoder unit, and connect the feature map after performing the projection processing to the feature map of an equal size in the decoder unit.

6: A tomography system comprising:
a tomography apparatus configured to acquire measurement data for creating a tomographic image of a subject; and
the image processing apparatus according to creating claim 1 configured to create the tomographic image based on the measurement data.

7: An image processing method for creating a tomographic image of a subject in a real space based on measurement data of the subject acquired by a measurement by using a tomography apparatus, the image processing method comprising:
a measurement image creation step of creating a measurement image in a measurement space different from the real space based on the measurement data; and
a CNN processing step of inputting the measurement image to a convolutional neural network, and creating the tomographic image by the convolutional neural network, wherein
the convolutional neural network includes:
an encoder unit configured to input the measurement image, update a feature map by a convolution operation performed by each of a plurality of convolutional layers, and gradually decrease a spatial size of the feature map;
a decoder unit provided at a subsequent stage of the encoder unit, and configured to update the feature map by a convolution operation performed by each of a plurality of convolutional layers, and gradually increase a spatial size of the feature map; and
a projection connection unit configured to perform projection processing from the measurement space to the real space on any one feature map in the encoder unit, and connect the feature map after performing the projection processing to the feature map of an equal size in the decoder unit.

8: The image processing method according to claim 7, further comprising:
a CNN training step of training the convolutional neural network by using the measurement image and the tomographic image of each of a plurality of subjects.

9: The image processing method according to claim 7, wherein in the CNN processing step, the projection processing from the measurement space to the real space is performed on the measurement image input to the encoder unit, and an image after performing the projection processing is added to an output image from the decoder unit.

10: The image processing method according to claim 7, wherein the projection connection unit is configured to perform the projection processing from the measurement space to the real space on the feature map of a maximum size in the encoder unit, and connect the feature map after performing the projection processing to the feature map of an equal size in the decoder unit.

11: The image processing method according to claim 7, wherein the projection connection unit is configured to perform the projection processing from the measurement space to the real space on each of the feature maps of all sizes in the encoder unit, and connect the feature map after performing the projection processing to the feature map of an equal size in the decoder unit.
